(19) United States
(12) Plant Patent Application Publication
(10) Pub. No.: US 2020/0113104 P1
(43) Pub. Date: Apr. 9, 2020

(54) Calla lily plant named 'Capitola'

(50) ;

(71) Applicant: Dümmen Group B.V., De Lier (NL)

(72) Inventor: Peter Beckman, Santa Cruz, CA (US)

(21) Appl. No.: 16/602,418

(22) Filed: Oct. 1, 2019

Related U.S. Application Data
(60) Provisional application No. 62/766,205, filed on Oct. 4, 2018.

(51) Int. Cl. A01H 6/56

(52) U.S. Cl. PLT/490

(57) ABSTRACT

‘Capitola’ is a new variety of calla lily plant having red-purple colored spathes that are positioned above the foliage. The flower stems are strong and long lasting. The leaves are green with no maculations present.

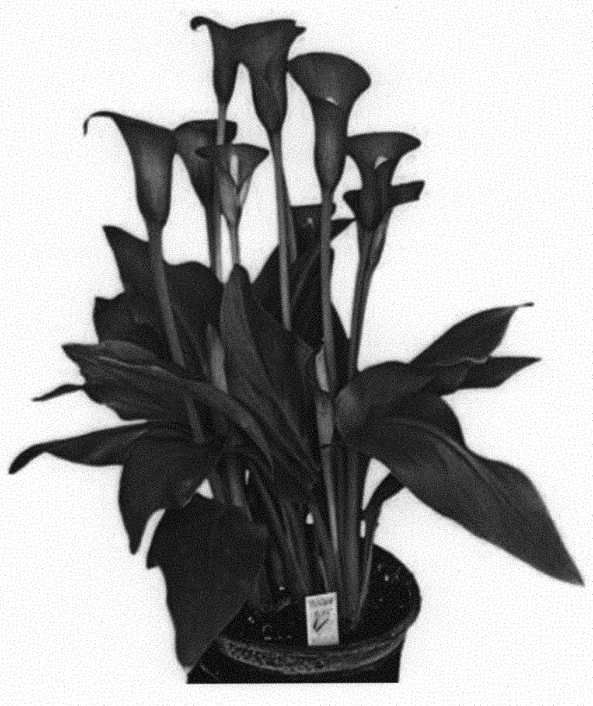

### CROSS REFERENCE TO RELATED APPLICATION

This application claims the benefit of U.S. Provisional Patent Application Serial No. 62/766,205, having a filing date of Oct. 4, 2018, and entitled “Varieties of Z. sprengeri Calla Lily Plants”, the entire disclosure of which is incorporated herein by reference.

Botanical classification: Zantedeschia sprengeri.

Varietal denomination: ‘Capitola’.

### BACKGROUND OF THE NEW PLANT

The present invention comprises a new and distinct cultivar of calla lily, botanically known as Zantedeschia sprengeri and hereinafter referred to by the cultivar name ‘Capitola’.

‘Capitola’ is a product of a planned breeding program and the new cultivar was selected from the crossing of an unnamed and unpatented Zantedeschia rehmannii seedling (female parent) with an unnamed and unpatented Zantedeschia elliottiana seedling (male parent). ‘Capitola’ was selected in Santa Cruz, Calif. as one flowering plant within the progeny of the stated cross.

Asexual reproductions of ‘Capitola’ have demonstrated that the combination of characteristics as herein disclosed for the new variety are retained and reproduced true to type through successive generations of asexual reproduction.

Color references are made to the 2001 R.H.S. Colour Chart of The Royal Horticultural Society of London, 4^{th} Edition, except where general color terms of ordinary significance are used.

The present invention has not been evaluated under all possible environmental conditions. The phenotype may vary with variations in environment without a change in the genotype of the plant.

The following traits have been repeatedly observed and determined to be basic characteristics of ‘Capitola’ which, in combination, distinguish this calla lily as a new and distinct cultivar:
1. Red-purple colored spathes;
2. Inflorescences are positioned above the foliage; and
3. Firm, long-lasting peduncles.

Table 1 provides a comparison of ‘Capitola’ to similar Zantedeschia sprengeri varieties ‘Garnet Glow’ (unpatented), ‘California Red’ (unpatented), and ‘Flirt’ (unpatented).

TABLE 1

| Comparison | Differing | Comparison | Present variety |
|---|---|---|---|
| variety | trait(s) | expression | expression |
| ‘Garnet | Peduncle | Early flower | Erect peduncles with |
| Glow’ | strength | nodding | no nodding observed |
| | | | at 21 days |
| ‘California | Spathe color | Red Group RHS | Between Red-Purple |
| Red’ | | 46A | Group RHS 59A- |
| | | | 59B |
| | Flowering time | 78 days to flower | 68 days to flower |
| ‘Flirt’ | Leaf maculation | Present | None present |

### BRIEF DESCRIPTION OF THE DRAWING

The accompanying photographic drawing illustrates the new variety, with the colors being as nearly true as is possible with color illustrations of this type.

### DESCRIPTION OF THE NEW PLANT

Type: Deciduous.
   Main use.—Potted plant.
   Flowering time.—68 days to flower.
   Disease/pest resistance.—Moderate tolerance to Pythium root rot and Erwinia bulb soft rot.
   Leaves.—Green, with no maculations present.
   Spathe.—Position: Above the foliage. Length: Short to medium. Width: Medium. Color: Throat spot: Absent. Upper surface: Between Red-Purple Group RHS 59A and 59B.
   Peduncle.—Strong and erect, with no nodding observed until day 21.

I claim:

1. A new and distinct variety of calla lily plant named ‘Capitola’ as herein described and illustrated.
